(19) United States
(12) Patent Application Publication
(10) Pub. No.: US 2026/0015563 A1
(43) Pub. Date: Jan. 15, 2026

(54) EDGE EFFECT SYSTEMS AND METHODS FOR FUNCTIONALIZED MICROFLUIDIC DEVICES

(71) Applicant: Xellar Ltd., Grand Cayman (KY)

(72) Inventors: Xin Xie, Lexington, MA (US); Xiaohua Qian, Sudbury, MA (US); Hardeep Singh, Arlington, MA (US)

(73) Assignee: Xellar Ltd., Grand Cayman (KY)

(21) Appl. No.: 19/092,471

(22) Filed: Mar. 27, 2025

Related U.S. Application Data
(63) Continuation of application No. PCT/US2023/025755, filed on Jun. 20, 2023.
(60) Provisional application No. 63/412,174, filed on Sep. 30, 2022.
(60) Provisional application No. 63/412,273, filed on Sep. 30, 2022.
(60) Provisional application No. 63/412,279, filed on Sep. 30, 2022.
(60) Provisional application No. 63/437,954, filed on Jan. 9, 2023.
(60) Provisional application No. 63/437,955, filed on Jan. 9, 2023.

(51) Int. Cl. C12M 3/06; C12M 1/12

(52) U.S. Cl. CPC C12M 23/16; C12M 25/14

(57) ABSTRACT

The present disclosure generally relates to microfluidics, and to systems and methods for controlling the flow of fluids. Certain aspects are generally directed to microfluidic channels that are parallel to each other, e.g., within a microfluidic interconnected region of a microfluidic device. In some embodiments, a fluid in a first microfluidic channel may be prevented from entering a second microfluidic channel due to a trench or other feature separating the channels. The trench may include features that at least partially prevent fluid from crossing. For example, the ends of the trench may be positioned such that fluids cannot access the ends, e.g., due to overhang regions between the trench and the microfluidic channels. This may keep the fluids pinned within the channels in some embodiments. Thus, for example, a fluid in a first microfluidic channel may be hardened to form a hydrogel, while the second microfluidic channel may remain free of the fluid and the hydrogel, due to the trench. Other embodiments are generally directed to devices containing such structures, methods or kits using such structures, or the like.

### RELATED APPLICATIONS

This application claims the benefit of U.S. Provisional Patent Application Ser. No. 63/412,174, filed Sep. 30, 2022, entitled “Microfluidic Devices Containing Hydrogels, and Techniques for Making and Using”; U.S. Provisional Patent Application Ser. No. 63/412,273, filed Sep. 30, 2022, entitled “Methods and Systems for Functionalizing Surfaces for Microfluidic Devices or Other Applications”; U.S. Provisional Patent Application Ser. No. 63/412,279, filed Sep. 30, 2022, entitled “Techniques and Systems for Creating Spatially Controlled Fluidic Flows in Surface Functionalized Microfluidic Devices”; U.S. Provisional Patent Application Ser. No. 63/437,954, filed Jan. 9, 2023, entitled “Edge Effect Systems and Methods for Functionalized Microfluidic Devices”; and U.S. Provisional Patent Application Ser. No. 63/437,955, filed Jan. 9, 2023, entitled “Pipette Interface Systems and Methods for Viscous Fluid Injection.” Each of these is incorporated herein by reference in its entirety.

### FIELD

The present disclosure generally relates to microfluidics, and to systems and methods for controlling the flow of fluids.

### BACKGROUND

In the body, cells adhere to the extracellular matrix, which is an intricate network of macromolecules organized in a cell/tissue specific manner. The ECM may assist cells in adhesion, communication, growth, movement, repair, or other cellular functions. The ECM includes a hydrogel such as collagen, which provides a mechanically stable structure that also serve as a reservoir for essential biomaterials that are used for cell growth and function. The hydrogel may include a crosslinked hydrophilic polymer network that does not dissolve in water.

However, ECM is difficult to model in vitro with certain types of microfluidic devices. For instance, it can be difficult to control the flow of fluids within microfluidic devices in order to create hydrogels representing the ECM. For example, when a hydrophilic fluid is added to the surface of a hydrophobic thermoplastic material such as polystyrene, the fluid tends to bead up due to surface tension between the two materials. This may create problems, for example, in causing a fluid to flow into desired locations within a microfluidic device. Improvements in systems and methods to control fluid flow of such fluids within microfluidic devices are therefore desirable.

### SUMMARY

The present disclosure generally relates to microfluidics, and to systems and methods for controlling the flow of fluids. The subject matter of the present disclosure involves, in some cases, interrelated products, alternative solutions to a particular problem, and/or a plurality of different uses of one or more systems and/or articles.

One aspect is generally drawn to an article. In one set of embodiments, the article comprises a substrate defining a first microfluidic channel having a first inlet and a first outlet, and a second microfluidic channel having a second inlet and a second outlet. In some cases, the first microfluidic channel and the second microfluidic channel may be positioned parallel and separated by a trench within a common interconnect region positioned between their respective inlets and outlets. In some embodiments, the trench has a length longer than a length of an interface between the first microfluidic channel and the second microfluidic channel in the common interconnect region.

In another set of embodiments, the article comprises a substrate defining a first microfluidic channel having a first inlet and a first outlet, and a second microfluidic channel having a second inlet and a second outlet. In some cases, the first microfluidic channel and the second microfluidic channel may be positioned parallel and separated by a trench within a common interconnect region positioned between their respective inlets and outlets. In some embodiments, an overhang region is positioned between the trench and the first microfluidic channel.

The article, in yet another set of embodiments, comprises a substrate defining a first microfluidic channel having a first inlet and a first outlet, and a second microfluidic channel having a second inlet and a second outlet. In some cases, the first microfluidic channel and the second microfluidic channel may be positioned parallel and separated by a trench within a common interconnect region positioned between their respective inlets and outlets. In some embodiments, wherein an end of the trench is in fluid communication with the first microfluidic channel only via the trench.

The article, in still another set of embodiments, comprises a substrate defining a first microfluidic channel and a second microfluidic channel. In some cases, the first microfluidic channel containing a hydrogel and the second microfluidic channel may be free of hydrogel. In certain embodiments, the first microfluidic channel and the second microfluidic channel may be positioned parallel within a common interconnect region such that an interface is present within the common interconnect region between the hydrogel in the first microfluidic channel and the second microfluidic channel. In some cases, the substrate may further define a trench positioned adjacent the interface. In one embodiment, the trench has a length longer than a length of an interface between the first microfluidic channel and the second microfluidic channel in the common interconnect region.

In another set of embodiments, the article comprises a substrate defining a first microfluidic channel and a second microfluidic channel, the first microfluidic channel containing a hydrogel and the second microfluidic channel being free of hydrogel, the first microfluidic channel and the second microfluidic channel positioned parallel within a common interconnect region such that an interface is present within the common interconnect region between the hydrogel in the first microfluidic channel and the second microfluidic channel, the substrate further defining a trench positioned adjacent the interface. In some cases, an overhang region is positioned between the trench and the first microfluidic channel. In some cases, an end of the trench is not in fluid communication with the first microfluidic channel.

Another aspect is generally drawn to a method. In one set of embodiments, the method comprises providing a substrate defining a first microfluidic channel having a first inlet and a first outlet, and a second microfluidic channel having a second inlet and a second outlet, and passing a fluid through the first microfluidic channel from the inlet towards the outlet, through the common interconnect region. In one embodiment, the first microfluidic channel and the second microfluidic channel may be positioned parallel within a common interconnect region positioned between their respective inlets and outlets. In some cases, the fluid is prevented from entering the second microfluidic channel via a trench in a wall of the common interconnect region. In certain embodiments, the trench has a length longer than a length of an interface between the first microfluidic channel and the second microfluidic channel in the common interconnect region.

In another set of embodiments, the method comprises providing a substrate defining a first microfluidic channel having a first inlet and a first outlet, and a second microfluidic channel having a second inlet and a second outlet, the first microfluidic channel and the second microfluidic channel positioned parallel within a common interconnect region positioned between their respective inlets and outlets, and passing a fluid through the first microfluidic channel from the inlet towards the outlet, through the common interconnect region, wherein the fluid is prevented from entering the second microfluidic channel via a trench in a wall of the common interconnect region. In some cases, an overhang region is positioned between the trench and the first microfluidic channel. In some cases, an end of the trench is not in fluid communication with the first microfluidic channel.

In another aspect, the present disclosure encompasses methods of making one or more of the embodiments described herein, for example, microfluidic devices containing trenches or other features for spatially controlling fluidic flows. In still another aspect, the present disclosure encompasses methods of using one or more of the embodiments described herein, for example, microfluidic devices containing trenches or other features for spatially controlling fluidic flows.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 illustrates a trench positioned between microfluidic channels at a common interconnect region, in accordance with one embodiment;

FIG. 2 illustrates an overhead view of a trench positioned between microfluidic channels at a common interconnect region, in accordance with another embodiment;

FIGS. 3A-3C illustrate various microfluidic chips having a plurality of repeat units, in yet another embodiment;

FIGS. 4A-4F illustrates a common interconnect region having three microfluidic channels, in yet other embodiments;

FIGS. 5A-5B illustrate various trench configurations, in still other embodiments;

FIG. 6 illustrates a closeup of an overhang region, in another embodiment;

FIG. 7 illustrates an example device having three microfluidic channels, in still another embodiment;

FIG. 8 illustrates microfluidic channels meeting at a common interconnect region, in accordance with certain embodiments;

FIGS. 9A-9C illustrate addition configurations of microfluidic devices having trenches, in yet other embodiments; and

FIG. 10 illustrates a microfluidic device having a vent connecting a microfluidic channel to a trench, in still another embodiment.

### DETAILED DESCRIPTION

The present disclosure generally relates to microfluidics, and to systems and methods for controlling the flow of fluids. Certain aspects are generally directed to microfluidic channels that are parallel to each other, e.g., within a microfluidic interconnected region of a microfluidic device. In some embodiments, a fluid in a first microfluidic channel may be prevented from entering a second microfluidic channel due to a trench or other feature separating the channels. The trench may include features that at least partially prevent fluid from crossing. For example, the ends of the trench may be positioned such that fluids cannot access the ends, e.g., due to overhang regions between the trench and the microfluidic channels. This may keep the fluids pinned within the channels in some embodiments. Thus, for example, a fluid in a first microfluidic channel may be hardened to form a hydrogel, while the second microfluidic channel may remain free of the fluid and the hydrogel, due to the trench. Other embodiments are generally directed to devices containing such structures, methods or kits using such structures, or the like.

One aspect of the present disclosure is generally directed to microfluidic devices able to contain cells, for example, that are positioned to be in contact with a hydrogel or another scaffold medium. In one set of embodiments, for instance, one or more cells may be cultured within a microfluidic device, e.g., on or in a hydrogel. The cells may thus be cultured within such a device in an environment that is more similar to their native environment (e.g., where the hydrogel or other scaffold medium may act as an extracellular matrix). In some cases, cells cultured in such conditions may exhibit more physiologically relevant behavior, e.g., due to improved or more biologically relevant cell-to-cell or cell-to-environment interactions. In addition, in certain embodiments, the cells may be cultured in a manner as to emulate various functions of specific organs, e.g., the microfluidic device may be used as an organ- on-a-chip device.

In some embodiments, a hydrogel or another scaffold medium may be contained within a microfluidic device, e.g., within a microfluidic channel defined in a substrate forming the microfluidic device. The hydrogel (or other scaffold medium) may partially or completely fill the microfluidic channel, and cells may be cultured on or in the hydrogel. In addition, in some embodiments, there may be one or more additional microfluidic channels. These may be used for various purposes, e.g., to deliver fluids such as cell media, provide nutrients, remove waste, or the like, to or from the hydrogel. Such channels may be free of hydrogel in certain embodiments. In addition, in some cases such as those discussed below, no physical barrier may be present between the hydrogel and fluid that may be present within the second microfluidic channel.

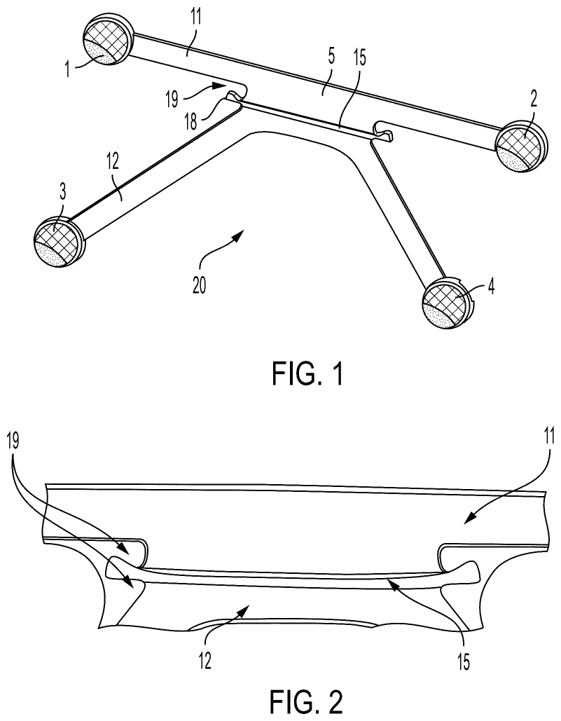

Referring now to FIG. 1, one non-limiting example embodiment of a microfluidic device is now described. In this figure, between inlet 1 and outlet 2 is first microfluidic channel 11, while between inlet 3 and outlet 4 is second microfluidic channel 12. In some cases, the inlets may be constructed and arranged to guide pipet tips towards the microfluidic channels, e.g., to facilitate the introduction of fluid therein. Non-limiting examples of such inlets can be seen in a provisional patent application filed on Jan. 9, 2023, entitled “Pipette Interface Systems and Methods for Viscous Fluid Injection,” U.S. Ser. No. 63/437,955, incorporated herein by reference in its entirety. First microfluidic channel 11 may be filled with a hydrogel or another scaffold medium, while second microfluidic channel 12 may be empty, e.g., such that during use of the microfluidic device, a fluid (e.g., cell media) can flow from inlet 3 to outlet 4 (or vice versa in some cases). This may be used, for example, to perfuse the cells within the microfluidic device, for example, contained on or within the hydrogel within first microfluidic channel 11.

In this figure, first microfluidic channel 11 and second microfluidic channel 12 come into fluidic contact via common interconnect region 5, e.g., such that a fluid could flow from one channel to the other if both channels were empty. In this region, first microfluidic channel 11 and second microfluidic channel 12 are positioned parallel to each other, e.g., such that there is no physical barrier that partially or completely separates the microfluidic channels from each other within the common interconnect region. For example, no pillars, columns, or other barriers may be present that separates first microfluidic channel 11 and second microfluidic channel 12.

Also shown here is trench 15, which may be positioned between first microfluidic channel 11 and second microfluidic channel 12 within common interconnect region 5. As discussed in more detail below, a trench may be positioned between a microfluidic channel and a second microfluidic channel, e.g., within a common interconnect region, for example, such as is shown here. The trench may be used in certain embodiments to separate or inhibit the flow of fluid from one microfluidic channel to another within the common interconnect region. Such a configuration may allow for separation of fluids to occur within the common interconnect region while avoiding the use of pillars, columns, bumps, phaseguides, ridges, or other barriers that may partially or completely block the common interconnect region. For instance, barriers that at least partially block the first microfluidic channel and the second microfluidic channel may also inhibit the ability of cells to access the cell media (e.g., to access nutrients, remove waste, etc.), and/or make it more difficult to study cells within the microfluidic device, etc., e.g., by making imaging of the cells more difficult.

As discussed herein, a trench may be used in some embodiments to separate fluids in one channel (e.g., a first microfluidic channel) from another channel (e.g., a second microfluidic channel). For instance, a fluid flowing through the first microfluidic channel may be inhibited from crossing the trench to reach the second microfluidic channel, e.g., such that the second microfluidic channel remains substantially free of the fluid. In some cases, the volume of fluid flowing through the first microfluidic channel may be controlled, e.g., to help inhibit crossing of the fluid to the second microfluidic channel. Thus, for example, if the first microfluidic channel contains a fluid containing a hydrogel (or another scaffold medium) precursor that is hardened to form a hydrogel, the presence of the trench may prevent the fluid from being able to flow into the second microfluidic channel at the common interconnect region. When the precursor is hardened to form a hydrogel, the hydrogel may be substantively contained within only the first microfluidic channel within the common interconnect region. A fluid flowing in the second microfluidic channel can interact with the hydrogel, without being blocked due to pillars, columns, or other physical barriers. Although other devices have used such physical barriers to separate the fluids in a common interconnect region, such physical barriers often interfere with the ability of fluids in one channel to subsequently interact with another channel within the common interconnect region. In contrast, a trench does not create a physical barrier between the channels.

In one set of embodiments, the trench may include features that are able to at least partially prevent fluid from crossing the trench. For example, the trench may be modified to prevent fluid from clinging to the edges or ends of the trench. One such example is illustrated in FIG. 1. As the non-limiting example in this figure shows, a trench in some embodiments may not be defined as the interface between the first microfluidic channel and the second microfluidic channel in the common interconnect region. Instead, the trench may have a length that is longer than the length of the interface, for example, as discussed herein. Thus, in various embodiments, a trench may extend outside of the common interconnect region, e.g., outside of the interface between the first microfluidic channel and the second microfluidic channel in the common interconnect region.

In certain embodiments, the ends of trench are not in fluid communication with one or both channels, other than via the trench itself. An example of such a configuration can be seen in FIG. 1 with end 18 of trench 15. The extended length of the trench, relative to the common interconnect region, may define a length that exceeds the length of the interface between the first microfluidic channel and the second microfluidic channel, and/or the length of the common interconnect region, thus defining an end of the trench that is not readily accessible from the channel, and accordingly difficult or impossible for fluids to reach, other than via the trench itself.

In some cases, the length of the trench may define an overhang region positioned between the trench, and one or both of the channels. A non-limiting example can be seen in FIG. 1 with overhang 19, positioned between end 18 of trench 15 and microfluidic channel 11. An overhang may have any suitable shape, e.g., being substantially rectangular and/or comprising one or more rounded end portions (for example, as is shown in FIG. 1). The overhang region may thus serve to prevent fluid from reaching the end portion of the trench, and thus preventing fluid from being able to cross the trench. In some cases, rounded end portions of the overhang region may prevent fluid from flowing into the trench, e.g., by capillary action.

In addition, some embodiments disclosed herein are generally directed to microfluidic channels having a polymer or other coating material, and a hydrogel or other scaffold medium, e.g., in contact with the polymer or other coating material. Optionally, cells may be grown on or in the hydrogel, e.g., as discussed herein. Other non-limiting examples of such microfluidic devices include those described in the following, each of which is incorporated herein by reference in its entirety: U.S. Pat. Apl. Ser. No. 63/412,174, filed on Sep. 30, 2022, entitled “Microfluidic Devices Containing Hydrogels, and Techniques for Making and Using”; U.S. Pat. Apl. Ser. No. 63/412,273, filed on Sep. 30, 2022, entitled “Methods and Systems for Functionalizing Surfaces for Microfluidic Devices or Other Applications”; and U.S. Pat. Apl. Ser. No. 63/412,279, filed on Sep. 30, 2022, entitled “Techniques and Systems for Creating Spatially Controlled Fluidic Flows in Surface Functionalized Microfluidic Devices.” Devices such as these may be used for culturing cells, or other applications such as those discussed herein.

The above discussion is a non-limiting example of certain embodiments that are generally directed to trenches that may be used to separate fluids, e.g., in a common interconnect region. However, other embodiments are also possible. Accordingly, more generally, various aspects of the invention are directed to various systems and methods for spatially controlling fluidic flows, e.g., within microfluidic devices.

One aspect, for example, is generally directed to a microfluidic device, e.g., having one or more microfluidic channels defined in a substrate. The substrate may have any suitable shape or configuration, including square, rectangular, circular, etc. In some cases, the substrate may include one or more layers of material. In certain cases, one or more layers of the substrate may be formed out of materials such as pressure-sensitive adhesives, or other materials, including any of those described herein. For instance, the microfluidic device may include one, two, three, four, or more layers, and one or more of the layers may contain or define one or more microfluidic channels therein. The layers can be bonded together using a variety of techniques, such as using pressure sensitive adhesives, or by thermal bonding, laser welding, etc. In addition, in some cases, larger channels, tubes, chambers, reservoirs, fluidic pathways, etc. may also be defined within a substrate, e.g., using one or more layers.

The microfluidic channels within the microfluidic device may have any configuration within the device, and there may be one or more than one such channel, which may independently be the same or different. A microfluidic channel may have any cross-sectional shape (circular, oval, triangular, irregular, square or rectangular, or the like) and can be covered or uncovered. The microfluidic channels may be used to move or process fluid within the substrate in any of a number of ways, for example, to allow fluids to flow from one or more inlets, through the microfluidic channel, to one or more outlets.

In some cases, the inlets may be constructed and arranged to guide pipet tips towards the microfluidic channels, e.g., to facilitate the introduction of fluid therein. Non-limiting examples of such inlets can be seen in a provisional patent application filed on Jan. 9, 2023, entitled “Pipette Interface Systems and Methods for Viscous Fluid Injection,” U.S. Ser. No. 63/437,955, incorporated herein by reference in its entirety.

In some cases, a microfluidic channel may have a maximum cross-sectional dimension of less than 10 mm, less than 8 mm, less than 7 mm, less than 6 mm, less than 5 mm, less than 3 mm, less than 2 mm, and in certain cases, less than 1 mm, less than 500 micrometers, less than 300 micrometers, less than 200 micrometers, less than 100 micrometers, less than 50 micrometers, less than 30 micrometers, less than 20 micrometers, less than 10 micrometers, less than 5 micrometers, etc. In addition, a microfluidic channel may have a maximum cross-sectional dimension of at least 5 micrometers, at least 10 micrometers, at least 20 micrometers, at least 30 micrometers, at least 50 micrometers, at least 100 micrometers, at least 200 micrometers, at least 300 micrometers, at least 500 micrometers, at least 1 mm, at least 2 mm, at least 3 mm, at least 5 mm, at least 6 mm, at least 7 mm, at least 8 mm, at least 10 mm, etc. Any combination of these is also possible. For instance, a microfluidic channel may have a maximum cross-sectional dimension of between 10 micrometers and 30 micrometers, between 100 micrometers and 500 micrometers, between 300 micrometers and 1 mm, or the like.

In some cases, all of the channels within a substrate or a layer may be microfluidic channels. However, in other cases, larger channels, tubes, chambers, reservoirs, fluidic pathways, etc. may also be present. Those of ordinary skill in the art will be familiar with microfluidic channels and systems and methods of making substrates containing microfluidic channels (and/or other channels).

In one set of embodiments, two, three, four, five, or more microfluidic channels may meet at a common interconnect region. In some cases, some or all of the microfluidic channels may be positioned to be parallel to each other within the common interconnect region, and in some cases, no physical barrier (e.g., pillars, columns, bumps, phaseguides, ridges, etc.) may be present within the common interconnect region that partially or completely separates the microfluidic channels from each other. Thus, for example, a fluid could flow from one channel within the common interconnect region to another channel within the common interconnect region if both channels were empty.

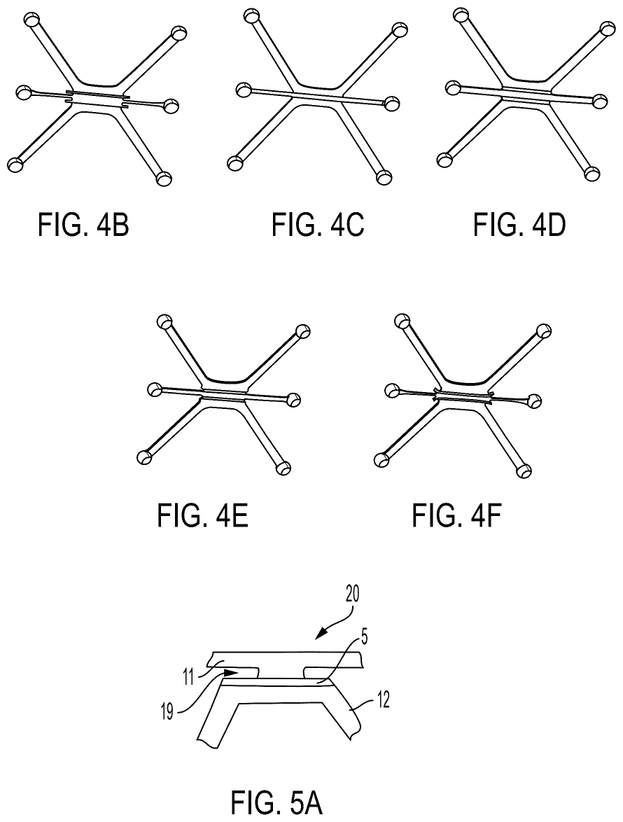

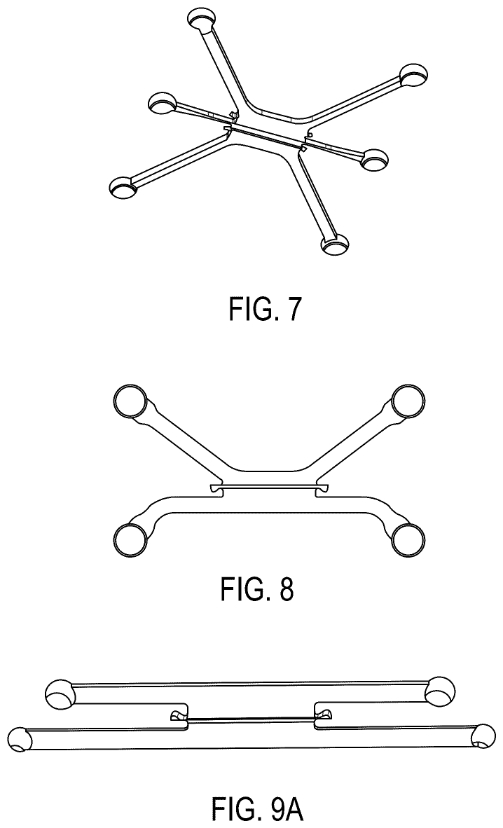

Non-limiting examples of a common interconnect region with two microfluidic channels are shown in FIGS. 1 and 8, while non-limiting examples of a common interconnect region with three microfluidic channels are shown in FIGS. 4A-4F and 7. For example, FIG. 4B shows a common interconnect region with 2 trenches, while FIG. 4C does not have trenches. FIG. 4D illustrates a common interconnect region having ridges present between various microfluidic channels that partially blocks fluidic communication between the microfluidic channels. In addition, combinations of features such as these can be combined in certain embodiments; for example, as is shown in FIGS. 4E and 4F with various embodiments containing both ridges and trenches.

The common interconnect region in some cases, may be treated as a microfluidic channel portion that is composed of two or more microfluidic channels that are in fluidic contact with each other and are generally positioned parallel to each other within the region, although the microfluidic channels may not necessarily be parallel outside of the common interconnect region. In a common interconnect region, the channels are not separated (e.g., by physical barriers such as pillars, columns, bumps, phaseguides, ridges, etc.), and the microfluidic channels can come into contact with each other such that the microfluidic channels in fluidic contact, e.g., to allow fluid flow between channels to occur within the common interconnect region. For example, a first microfluidic channel may have a first inlet and a first outlet, and a second microfluidic channel may have a second inlet and a second outlet, and the first and second microfluidic channels may come into contact and be positioned parallel to each other within the common interconnect region between their respective inlets and outlets (although outside of the common interconnect region, they may or may not also be parallel).

As a non-limiting example, as discussed herein, a first microfluidic channel may contain a hydrogel or other scaffold medium, while a second microfluidic channel may contain a fluid (e.g., cell media), and within the common interconnect region, the fluid is able to come into direct contact with the hydrogel or other scaffold medium, e.g., without having to circumvent a physical barrier, such as a pillar or a column. Accordingly, in certain embodiments, there may be a barrierless interface in a common interconnect region between a first fluid or medium in a first microfluidic channel (for example, a hydrogel or other scaffold medium), and a second fluid or medium in a second microfluidic channel (for example, cell media). For instance, in some embodiments, no interface material or physical barrier separating the first fluid or medium from the second fluid or medium may be present. Thus, for example, a hydrogel or other scaffold medium may partially fill the common interconnect region, for example, such that at least 20%, at least 30%, at least 40%, at least 50%, at least 60%, at least 70%, at least 80%, and/or no more than 80%, no more than 70%, no more than 60%, no more than 50%, no more than 40%, no more than 30%, or no more than 20% of any cross-section of the common interconnect region is not filled with the hydrogel or other scaffold medium. In some embodiments, the hydrogel (or other scaffold medium) partially fills the common interconnect region such that the hydrogel does not prevent bulk fluid flow through at least a portion of the common interconnect region.

In some cases, at least a portion, or all, of the common interconnect region may be substantially straight. In addition, in certain embodiments, the microfluidic channels are positioned within the common interconnect region to be substantially parallel to each other. The parallel microfluidic channels can be used to define an imaginary channel axis that passes through the common interconnect region, e.g., in a direction defined by the direction that the parallel microfluidic channels are oriented. However, in certain cases, one or more of the microfluidic channels may be at an angle relative to other microfluidic channels within the common interconnect region.

In some embodiments, the common interconnect region may have a longest dimension along the channel axis (if present) of at least 1 mm, at least 2 mm, at least 3 mm, at least 4 mm, at least 5 mm, at least 6 mm, at least 7 mm, at least 8 mm, at least 9 mm, at least 10 mm, etc. In addition, the common interconnect region may have a longest dimension along the channel axis of no more than 10 mm, no more than 9 mm, no more than 8 mm, no more than 7 mm, no more than 6 mm, no more than 5 mm, no more than 4 mm, no more than 3 mm, no more than 2 mm, no more than 1 mm, etc. Combinations of these are also possible in other embodiments. For example, the common interconnect region may have a longest dimension of between 5 mm and 7 mm, between 4 mm and 8 mm, between 2 mm and 6 mm, etc.

In certain embodiments, the common interconnect region may have a maximum cross-sectional dimension, or a maximum dimension orthogonal to the channel axis (if present), of at least 100 micrometers, at least 200 micrometers, at least 300 micrometers, at least 500 micrometers, at least 1 mm, at least 2 mm, at least 3 mm, at least 5 mm, at least 10 mm, at least 20 mm, at least 30 mm, at least 50 mm, at least 100 mm, etc. In addition, in certain embodiments, the common interconnect region may have maximum dimensions of no more than 100 mm, no more than 50 mm, no more than 30 mm, no more than 20 mm, no more than 10 mm, no more than 5 mm, no more than 3 mm, no more than 2 mm, no more than 1 mm, no more than 500 micrometers, no more than 300 micrometers, no more than 200 micrometers, no more than 100 micrometers, etc. In addition, combinations of any of these are also possible. For example, a common interconnect region may have maximum dimensions of between 100 micrometers and 300 micrometers, between 5 mm and 10 mm, between 500 micrometers and 2 mm, or the like.

In one set of embodiments, two or more microfluidic channels within a common interconnect region may be separated using a trench, e.g., on or in a wall of the common interconnect region. Non-limiting examples of trenches include those described in U.S. Pat. Apl. Ser. No. 63/412,279, filed on Sep. 30, 2022, entitled “Techniques and Systems for Creating Spatially Controlled Fluidic Flows in Surface Functionalized Microfluidic Devices,” incorporated herein by reference. Additional non-limiting examples of trenches are shown in FIGS. 7 and 8.

More than one trench may also be present in some cases, e.g., on opposed surfaces within the common interconnect region. Without wishing to be bound by any theory, it is believed that a fluid flowing in a channel may be attracted to a channel surface, e.g., due to similar hydrophilicities (e.g., if both are relatively hydrophilic or hydrophobic) and/or capillary action, which may facilitate the flow of the fluid within the channel. However, it may be difficult in certain embodiments for such a fluid to be able to cross a trench, e.g., if the volume of fluid is not too great. For example, the trench may exhibit a different hydrophilicity (e.g., one that does not promote attraction with the fluid), and/or the shape of the trench may discourage the fluid from being able to cross, e.g., due to the dimensions of the trench. In some embodiments, the trench may facilitate the flow of fluid through one channel within the common interconnect region, for example, without the fluid flowing into another channel within the common interconnect region. In addition, in certain embodiments, the trench may be treated, e.g., as discussed herein, to render it more hydrophilic or hydrophobic. For example, a coating material, such as a hydrophobic polymer, may be coated on at least a portion of the trench.

Accordingly, in some embodiments, a trench may be positioned within a common interconnect region between a first microfluidic channel and a second microfluidic channel. The trench may run along the length of the common interconnect region in some embodiments, e.g., to separate the two channels. Such a trench may thus provide physical separation of the channels, e.g., without the use of physical barriers (e.g., pillars, columns, bumps, phaseguides, ridges, etc.) to separate the channels. Such trenches are also discussed in more detail in U.S. Pat. Apl. Ser. No. 63/412,174, filed on Sep. 30, 2022, entitled “Microfluidic Devices Containing Hydrogels, and Techniques for Making and Using,” incorporated herein by reference in its entirety. However, it should be understood that in other embodiments, a trench may be used in conjunction with pillars, columns, bumps, phaseguides, ridges, or other barriers.

The trench may have any suitable dimensions or shape within the common interconnect region. For example, the trench may be substantially straight, or the trench may be bent or curved in certain embodiments. In some cases, the trench may have a length comparable to the length of the common interconnect region. In some embodiments, the trench may have a maximum length of at least 1 mm, at least 2 mm, at least 3 mm, at least 4 mm, at least 5 mm, at least 6 mm, at least 7 mm, at least 8 mm, at least 9 mm, at least 10 mm, etc. In some embodiments, the maximum length may no more than 10 mm, no more than 9 mm, no more than 8 mm, no more than 7 mm, no more than 6 mm, no more than 5 mm, no more than 4 mm, no more than 3 mm, no more than 2 mm, no more than 1 mm, etc. Combinations of these are also possible in other embodiments. For example, the length of the trench may be between 5 mm and 7 mm, between 4 mm and 8 mm, between 2 mm and 6 mm, etc.

In some embodiments, a trench may have a cross-sectional dimension of at least 10 micrometers, at least 20 micrometers, at least 30 micrometers, at least 50 micrometers, at least 100 micrometers, at least 200 micrometers, at least 300 micrometers, at least 500 micrometers, at least 1 mm, at least 2 mm, at least 3 mm, at least 5 mm, at least 10 mm, etc. In addition, in some embodiments, the trench may have a cross-sectional dimension of no more than 10 mm, no more than 5 mm, no more than 3 mm, no more than 2 mm, no more than 1 mm, no more than 500 micrometers, no more than 300 micrometers, no more than 200 micrometers, no more than 100 micrometers, no more than 50 micrometers, no more than 30 micrometers, no more than 20 micrometers, no more than 10 micrometers, etc. In addition, combinations of any of these are also possible, e.g., a trench may have a cross-sectional dimension of between 100 micrometers and 300 micrometers, between 200 micrometers and 1 mm, between 500 micrometers and 3 mm, etc. The trench may have a constant cross-sectional dimension, or a cross-sectional dimension that varies in some embodiments.

In addition, the trench may have any suitable depth. The depth may be independent of the cross-sectional dimension. In some embodiments, the depth may be at least 1 micrometer, at least 2 micrometers, at least 3 micrometers, at least 5 micrometers, at least 10 micrometers, at least 20 micrometers, at least 30 micrometers, at least 50 micrometers, at least 100 micrometers, at least 200 micrometers, at least 300 micrometers, at least 500 micrometers, at least 1 mm, at least 2 mm, at least 3 mm, at least 5 mm, at least 10 mm, at least 20 mm, at least 30 mm, at least 50 mm, etc. In addition, in some cases, the depth may be no more than 50 mm, no more than 30 mm, no more than 20 mm, no more than 10 mm, no more than 5 mm, no more than 3 mm, no more than 2 mm, no more than 1 mm, no more than 500 micrometers, no more than 300 micrometers, no more than 200 micrometers, no more than 100 micrometers, no more than 50 micrometers, no more than 30 micrometers, no more than 20 micrometers, no more than 10 micrometers, no more than 5 micrometers, no more than 3 micrometers, no more than 2 micrometers, no more than 1 micrometer, etc. In addition, combinations of any of these are also possible in certain embodiments. For instance, the trench may have a depth of between 2 mm and 3 mm, between 1 mm and 10 mm, between 100 micrometers and 2 mm, etc. The trench may have a constant depth, or a depth that varies in some cases.

In some aspects, a trench may include features that are able to at least partially prevent fluid from crossing the trench. Without wishing to be bound by any theory, it is believed that under certain conditions, a fluid may be able pass over the trench by clinging to the edges or ends of the trench that are positioned between the channels, for example, due to surface tension or edge effects. Accordingly, even though a trench can be used to prevent fluid from crossing from one channel to another within the common interconnect region, the trench may not be able to fully prevent the fluid from crossing under certain conditions.

Thus, in accordance with certain embodiments, the trench may be modified to prevent fluid from clinging to the edges or ends of the trench. For instance, in some cases, the trench may be constructed and arranged to have a length that is longer than the length of the interface between the first microfluidic channel and the second microfluidic channel in the common interconnect region. This may be useful, for example, in ensuring that one or both ends of the trench are locate at a spaced distance away from a microfluidic channel containing a fluid, e.g., a hydrogel solution, which may be useful in helping to prevent the fluid from crossing from one channel to another within the common interconnect region via an end of the trench.

Accordingly, in some embodiments, the trench may have a length that is longer than the length of the interface. For example, the trench may be at least 0.1 mm, at least 0.2 mm, at least 0.3 mm, at least 0.4 mm, at least 0.5 mm, at least 0.6 mm, at least 0.7 mm, at least 0.8 mm, at least 0.9 mm, at least 1 mm, at least 2 mm, at least 3 mm, at least 4 mm, at least 5 mm, at least 6 mm, at least 7 mm, at least 8 mm, at least 9 mm, or at least 10 mm longer than the length of the interface between the first and second microfluidic channels within the common interconnect region. The trench may be positioned such that it is symmetric or asymmetric with respect to the interface region. For example, one or both of the ends of the trench may be positioned outside of the common interconnect region, e.g., outside of the interface between the first microfluidic channel and the second microfluidic channel in the common interconnect region, or only one end of the trench may be positioned outside of the common interconnect region, etc.

Accordingly, in some embodiments, one or both ends of a trench may be positioned such that they are not in fluid communication with one or both channels, other than via the trench itself. For instance, an end of the trench may be positioned at least 0.1 mm, at least 0.2 mm, at least 0.3 mm, at least 0.4 mm, at least 0.5 mm, at least 0.6 mm, at least 0.7 mm, at least 0.8 mm, at least 0.9 mm, at least 1 mm, at least 2 mm, at least 3 mm, at least 4 mm, at least 5 mm, etc. from one or both of the first microfluidic channel and/or the second microfluidic channel. In some cases, an end of the trench is positioned such that it is not readily accessible from the channel, other than through the trench itself, and accordingly the end of the trench may be difficult or impossible for fluids to reach. Thus, the end of the trench may not be in fluid communication with one or both microfluidic channels. This may help to prevent a fluid (such as a fluid containing a hydrogel) from being able to cross the trench, e.g., due to edge effects.

In certain embodiments, a trench may define an overhang region positioned between the trench, and one or both of the channels. The overhang region may have any suitable shape. For instance, the overhang region may be substantially rectangular. In another example, an overhang region may include one or more rounded end portions. One non-limiting example of an overhang region can be seen in FIG. 6.

In some cases, a trench may define two overhang regions on an end of the trench, e.g., a first overhang region between the end of the trench and a first microfluidic channel, and a second overhang region between the end of the trench and a second microfluid channel. There may also be three or four overhang regions in certain embodiments for a given trench. If more than one overhang region is present, the overhang regions may independently have the same or different shapes. An overhang region, in certain cases, may be able to prevent fluid in a channel from reaching the end portion of the trench, and accordingly helping to prevent fluid from being able to cross the trench.

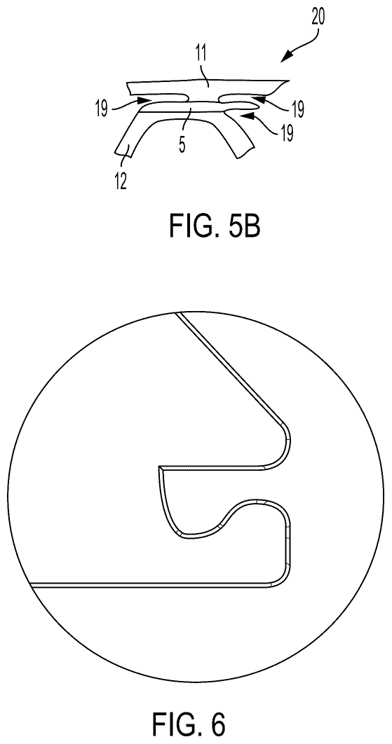

Non-limiting examples of such trench geometries can be seen in the figures. For instance, in FIG. 2, a trench is positioned between two microfluidic channels, defining two overhang regions on each end of the trench, or four overhang regions total. Two of these overhang regions is marked as 19 in this figure. However, it should be understood that there may be more or fewer overhang regions present. As a non-limiting example, in FIG. 5A, in microfluidic device 20, an overhang region 19 may be defined between trench 5 and first microfluidic channel 11, but there may be no overhang region between trench 5 and second microfluidic channel 12. As another example, multiple trenches with multiple such overhang regions are shown in FIG. 7 in a system with three microfluidic channels.

In addition, it should be noted that in various embodiments, the trench and/or the trench ends may be symmetric, or asymmetric in some cases. For example, in FIG. 5B, the ends of the trench are not symmetrically arranged.

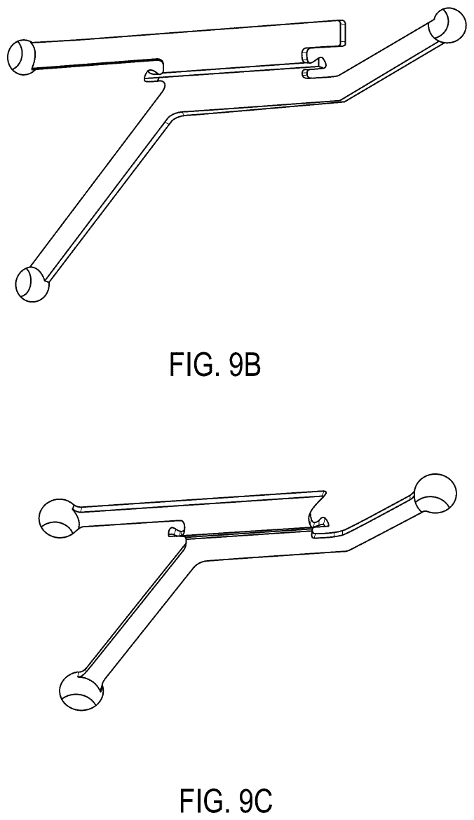

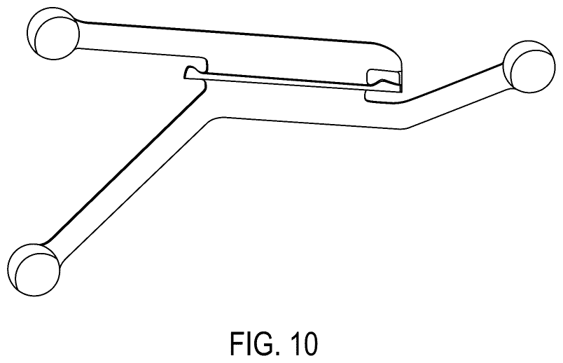

In addition, in one set of embodiments, a microfluidic channel may pass between a single port and a microfluidic interconnect region, e.g., there may not necessarily be both an inlet and an outlet of a microfluidic channel. One example of such a configuration is shown in FIG. 9B. In some cases, a vent may be present at an end of the microfluidic channel, e.g., to allow air or other gases to flow out of the microfluidic channel, for example, when the channel is being filled with a fluid. In some embodiments, the vent may connect an end of the microfluidic channel to a second microfluidic channel, and/or to a trench (if present). One non-limiting examples of such a vent is shown in FIG. 10. However, in other cases, no vent may be present.

In one set of embodiments, the microfluidic channels may have any suitable configuration. If more than one microfluidic channel is present, the channels may independently have the same or different lengths. In some cases, one or more microfluidic channels may intersect, for example, in a T, Y, or a + intersection, or within a common interconnect region such as described herein, etc. Other types of intersections are also possible. A microfluidic channel, in some cases, may be substantially straight between an inlet and an outlet. In addition, in some cases, a microfluidic channel may have one, two, or more bends, curves, or the like between an inlet and an outlet. (As a non-limiting example, as is shown in FIG. 1, microfluidic channel 12 has two bends between inlet 3 and outlet 4.) If more than one microfluidic channel is present, the microfluidic channels may independently have the same or different configurations. In some cases, there may be 0, 1, 2, or more intersections with other microfluidic channels between an inlet and an outlet of the microfluidic channel.

Non-limiting examples of microfluidic channels with different configurations include those shown in FIGS. 8 and 9A-9C. For instance, in FIG. 9A, two substantially straight microfluidic channels passing between an inlet and an outlet may connect at a common interconnect region, separated by an optional trench in some embodiments.

In addition, it should be understood that a microfluidic channels may not necessarily pass between an inlet and an outlet. For instance, one of the microfluidic channels may have only a single port, which can be used as an inlet and/or an outlet (one non-limiting example is shown in FIG. 9B). In some cases, for instance, fluid may pass through a common interconnect region from an inlet of a first microfluidic channel to an outlet of a second microfluidic channel.

A microfluidic channel may have any suitable pathlength, e.g., length along the channel as fluid flows between an inlet and an outlet of the channel. If more than one microfluidic channel is present, the microfluidic channels may independently have the same or different pathlengths. For instance, in some embodiments, a microfluidic channel may have a pathlength of at least 1 mm, at least 2 mm, at least 3 mm, at least 4 mm, at least 5 mm, at least 6 mm, at least 7 mm, at least 8 mm, at least 9 mm, at least 10 mm, at least 12 mm, at least 15 mm, at least 20 mm, etc. In some embodiments, the maximum pathlength may no more than 20 mm, no more than 15 mm, no more than 12 mm, no more than 10 mm, no more than 9 mm, no more than 8 mm, no more than 7 mm, no more than 6 mm, no more than 5 mm, no more than 4 mm, no more than 3 mm, no more than 2 mm, no more than 1 mm, etc. Combinations of these are also possible in other embodiments. For example, the length of a microfluidic channel may be between 5 mm and 7 mm, between 4 mm and 8 mm, between 2 mm and 6 mm, etc.

In addition, in one set of embodiments, a coating material may be present on one or more walls defining a microfluidic channel, for example, to alter the hydrophilicity of the walls, although in some embodiments, no coating materials may be present. For example, the coating material may increase or decrease the hydrophilicity of at least one of the walls defining a microfluidic channel. Different walls of the microfluidic channel may independently have the same or different hydrophilicities, for example, by coating different walls with different coating materials (or no coating material). Without wishing to be bound by any theory, it should be understood that, due to the small and cramped nature of the microfluidic channels, a fluid within a microfluidic channel may interact with the walls of the microfluidic channels, which can affect the flow properties of the fluid flowing through the channel. Thus, in some embodiments, the hydrophilicities of the walls forming a microfluidic channel may affect the flow of fluid through the channel.

For example, in one set of embodiments, a fluid containing a polymer or other suitable coating material may be flowed through a microfluidic channel, and in some cases, the fluid may be constrained to prevent it from entering other microfluidic channels. For instance, in some cases, a fluid may enter a first microfluidic channel in a common interconnect region, but due to the presence of adhesive or other feature that masks other microfluidic channels within the common interconnect region, the fluid is not able to enter the masked channels. In some cases, the coating material may be deposited onto one or more walls containing the fluid. This may be useful, for example, for altering the hydrophilicity of the walls, for creating a surface for adhering other materials to the walls, for altering the opacity of the walls, or other applications. In addition, other methods of adding a coating material may be used, for example, dip coating or drop casting.

Non-limiting examples of polymers that may be deposited onto one or more walls of a microfluidic channel, e.g., to form a coating thereon, include polyvinylpyrrolidone (PVP), poly(ethylene glycol) (PEG), poly(vinyl alcohol) (PVA), polylysine, or the like. In addition, in some cases, the coating materials may include other materials, in addition to or instead of polymers such as these, for example, ECM attachment factor. In some cases, coating materials, including polymers such as these, may be used to alter or increase the hydrophilicity of the microfluidic channel. In some cases, the increased hydrophilicity may be determined as a change in water contact angle, or by applying 2 microliters of water to a surface of the hydrophilic coating, and measuring a spread of water onto the surface of at least 10 mm².

In one set of embodiments, a hydrogel or other scaffold medium may be positioned on, adjacent to, or attached to the coating, e.g., such that the coating is positioned or located between the hydrogel and a wall of the microfluidic channel. The hydrogel (or other scaffold medium) may be applied, for example, by flowing a fluid containing a hydrogel or other scaffold medium precursor through a microfluidic channel, and treating the precursor to form the hydrogel or other scaffold medium. For example, the hydrogel precursor may be caused to harden to form a hydrogel. In some cases, the fluid containing the precursor may be a hydrophilic fluid, such as water, saline, or buffer, and in certain embodiments, the fluid may be preferentially attracted to a hydrophilic coating material, e.g., that may be present on one or more walls of a microfluidic channel. Examples of hydrophilic coatings include any of those described herein. In some cases, the fluid containing the precursor may preferentially be contained within a first microfluidic channel (e.g., within a common interconnect region as describe herein), without entering other microfluidic channels. Upon treatment (e.g., hardening), the resultant hydrogel (or other scaffold medium) may be positioned on the coating material within the first microfluidic channel, while other microfluidic channels may be substantially free of the hydrogel or other scaffold medium.

Non-limiting examples of hydrogels (e.g., that can be used as an extracellular matrix for cells) include collagen (e.g., Type I collagen, Type II collagen, Type III collagen, etc.), Matrigel®, methacrylated gelatin (Gel-MA), fibrin, alginate, hyaluronic acid, polyacrylamide, poly(ethylene glycol), poly(vinyl alcohol), agarose, agar, chitosan, poly(RADARADARADARADA) (PuraMatrix), poly(AEAEAKAKAEAEAKAK) (EAK16), poly(KLDLKLDLKLDL) (KLD12), or the like. In addition, more than one of these and/or other materials may be present in a hydrogel in certain instances. The collagen may arise from any suitable source, e.g., bovine collagen, rat collagen, fish (marine) collagen, chicken collagen, porcine collagen, sheep collagen, or the like. Other hydrogels will be known by those of ordinary skill in the art. In some embodiments, hydrogels such as these can be formed by flowing a fluid containing a hydrogel precursor, and causing the precursor to form the hydrogel, for example, using a change in temperature (e.g., cooling the device), exposure to ultraviolet radiation, exposure to a chemical, or the like.

In addition, other scaffold media can be used in certain embodiments, e.g., instead of or in addition to a hydrogel as discussed herein. Thus, it should be understood that hydrogels are described herein by way of example only. Non-limiting examples of other scaffold media that may be used in certain embodiments include paraffin, waxes, or the like. These may be added, for example, by flowing a fluid containing a scaffold medium precursor into a microfluidic channel within the device, and treating the precursor to form the scaffold medium within the device. For example, a paraffin or a wax may be introduced into a device at a temperature where the material is liquid, and treated (e.g., cooled) to solidify the medium within the microfluidic device.

In addition, in one set of embodiments, the scaffold medium may be substantially transparent, e.g., to allow for imaging of cells, such as is described herein. As a non-limiting example, in one embodiment, a hydrogel comprising collagen may be used.

According to one set of embodiments, the hydrogel or other scaffold medium may be exposed to cells, which may be grown or cultured on or in the hydrogel or other scaffold medium in some embodiments. Any suitable technique may be used to apply the cells. In some cases, for instance, the cells may be suspended in solution, which is flowed past the hydrogel or other scaffold medium, e.g., within the common interconnect region, and allowed to incubate there to promote attachment of the cells. In some cases, this process may occur over a period of at least 24 hours, or other suitable times. In addition, in some cases, the cells may be mixed with a fluid containing a hydrogel precursor or other scaffold medium precursor, e.g., prior to introduction to the microfluidic device. The cells may then be incubated and allowed to become embedded within the hydrogel or other scaffold medium. Those of ordinary skill in the art will be familiar with techniques for attaching cells to a suitable scaffold medium. Without wishing to be bound by any theory, it is believed that culturing cells on or in such an scaffold medium, e.g., a hydrogel, may more closely approximate the conditions that the cells naturally grow in, e.g., as opposed to a 2-dimensional surface. Accordingly, such cells may respond more similarly and appropriately when cultured in a 3-dimensional environment, such as a hydrogel.

Examples of cells that may be cultured on or in a hydrogel or other scaffold medium include, but are not limited to, mammalian cells such as human cells. Specific non-limiting examples include fibroblasts, lung cells, liver cells, fat cells, kidney cells, intestinal cells, brain cells, epithelial cells, endothelial cells, stromal cells, immune cells, or the like. In some cases, the cells may be stem cells, such as pluripotent stem cells, totipotent stem cells, multipotent stem cells, etc. Other cell types are also possible. In some cases, more than one type of cell may be present, e.g., liver cells and fibroblasts. In addition, in certain embodiments, the cells may produce organoids, tubes, or other 3-dimensional structures, e.g., depending on the cells being cultured.

In some cases, the cells may be cultured within the microfluidic device, for example, within a common interconnect region. In some cases, for instance, in a common interconnect region, a first microfluidic channel may contain a hydrogel or other scaffold medium, and cells that are in contact with the hydrogel or other scaffold medium. The common interconnect region may also comprise a second microfluidic channel that can contain a fluid (for example, cell media) that is able to maintain the cells within the hydrogel. Non-limiting examples of cell media include MEM, DMEM, RPMI, IMDM, F-10, or the like. Those of ordinary skill in the art will be able to select appropriate cell media, e.g., based on the type of cells that are present within the common interconnect region. In some cases, fluid is able to flow in and out of the common interconnect region, e.g., as the hydrogel (or other scaffold medium) may only partially fill the common interconnect region, thereby allowing fluid flow to occur through the common interconnect region. In addition, in some cases, the fluid may be in direct contact with the hydrogel or other scaffold medium, e.g., without having to circumvent a pillar, column, or other physical barrier. Thus, in some embodiments, there may be a barrierless interface between the hydrogel or other scaffold medium and a fluid (e.g., cell media) within the common interconnect region. This may allow the cells to be perfused by the cell media, e.g., to provide nutrients or dissolved gases, remove waste, or the like.

In addition, according to one set of embodiments, a first microfluidic channel and a second microfluidic channel may meet at a common interconnect region where the channels are positioned parallel within the common interconnect region. As previously discussed, there may optionally be a trench positioned between the first microfluidic channel and the second microfluidic channel at the common interconnect region. In some cases, the first microfluidic channel may be a straight channel between a first inlet and an outlet, while the second microfluidic channel may include bends on either side of the common interconnect region between a second inlet and a second outlet, thereby forming a K-shaped structure. A non-limiting example of such a structure can be seen in FIG. 1. In some cases, as discussed herein, one or more of the channels may contain a hydrogel or other scaffold medium, e.g., such that the hydrogel or other scaffold medium does not completely fill the common interconnect region and a fluid can pass between an inlet and an outlet through a microfluidic channel within the common interconnect region, e.g., in a microfluidic channel that is free of the hydrogel or other scaffold medium.

In one set of embodiments, there may be a plurality of repeat units on a substrate, e.g., repeat units including one or more microfluidic channels or common interconnect regions, such as those described herein. For instance, there may be at least 3, at least 5, at least 10, at least 20, at least 30, at least 40, at least 50, at least 60, at least 70, at least 80, at least 90, at least 100, at least 150, at least 200, at least 300, at least 500, at least 1000, at least 1500, etc. repeat units on a substrate. The repeat units may be all identically oriented, or they may be differently oriented (e.g., rotated, flipped, etc.) in certain embodiments. In addition, in some cases, two, three, or more types of repeat units may be present on a substrate, e.g., having dissimilar configurations.

In some embodiments, the repeat units may be regularly arranged on a substrate. For instance, the repeat units may be arranged as a square, a rectangle, a circle, a hexagonal configuration, or the like. In addition, the repeat units may be irregularly arranged in certain cases. As an example, the repeat units may be arranged in a 2×n configuration, where n is 1, 2, 3, 4, 5, 6, 7, 8, 9, 10, or the like. As another non-limiting example, the repeat units may be arranged in a 3×n configuration, a 4×n configuration, a 6×n configuration, an 8×n configuration, a 12×n configuration, a 16×n configuration, or the like. For example, the repeat units may be arranged in a 6×6 configuration, an 8×8 configuration, or the like, a 16×16 configuration, or the like.

The microfluidic channels, according to one set of embodiments, may be contained with a substrate having dimensions comparable to a microscope slide, e.g., arranged into a plurality of repeat units on the substrate. For example, the substrate may have dimensions of 75 mm×25 mm, 75 mm×26 mm, 46 mm×28 mm, 46 mm×27 mm, 75 mm×38 mm, 76 mm×51 mm, 76 mm×52 mm, etc. In some cases, such dimensions may vary somewhat (for example, by +/−1 mm, +/−2 mm, or +/−5 mm, etc.), e.g., to allow for manufacturing tolerances or the like. Such dimensions may be useful in some embodiments, e.g., to interface with laboratory equipment able to handle microscope slides.

In another set of embodiments, the microfluidic channels may be contained with a substrate having dimensions comparable to a microwell plate, e.g., one having ANSI dimensions of 128 mm×85 mm, e.g., arranged into a plurality of repeat units on the substrate. In some cases, the dimensions may vary somewhat (for example, by +/−1 mm, +/−2 mm, or +/−5 mm, etc.), e.g., to allow for manufacturing tolerances or the like. Such dimensions may be useful in some embodiments, e.g., to interface with laboratory equipment, such as plate readers or liquid handling robots that are able to handle microwell plates. In addition, in some embodiments, one or more inlets and/or outlets may be positioned within the substrate to match the locations of wells on a microwell plate, e.g., the center locations of the wells on a 24-well standard microplate, a 48-well standard microplate, a 96-well standard microplate, a 384-well standard microplate, or a 1536-well standard microplate, etc.

The substrate may be formed from any suitable materials. In some cases, the substrate may be formed from one, two, three, four, five, or more layers of materials, which may independently be the same or different. For instance, a layer within the substrate may comprise glass or a polymer. Non-limiting examples of polymers include polystyrene, polycarbonate, polymethylmethacrylate (PMMA), polycarbonate, polypropylene, cyclic olefin polymers (COP), cyclic olefin copolymers (COC), polyethylene terephthalate (PET), or the like. For example, an outer or end layer of the substrate may comprise glass or polymer, which may be useful for protecting internal components of the microfluidic device. In addition, as discussed herein, one or more of the layers of the microfluidic channel may be chosen to be substantially transparent.

In some cases, the substrate, or one or more layers, may be chosen to be substantially transparent, for example, to allow for imaging of the common interconnect region (for example, cells within the common interconnect region), or other locations within the substrate. In some embodiments, the entire substrate may be substantially transparent. A variety of techniques may be used for imaging, including light or optical microscopy, confocal microscopy, fluorescence microscopy, microwell plate readers, or the like. Those of ordinary skill in the art will be aware of other suitable imaging techniques. In some cases, multiple locations within a microfluidic device may be studied, e.g., sequentially and/or simultaneously. For example, in some embodiments, the microfluidic device may contain a plurality of repeat units that can be independently determined. In certain embodiments, fluid (e.g., cell media) may be flowed through a common interconnect region (e.g., to perfuse cells, etc., as discussed herein) during imaging (for example, uni- or bidirectionally), although in other cases no such flow may occur during imaging.

In one set of embodiments, microfluidic devices such as those described herein may be used for the study of cells or other constructs, such as organoids, tubes, or other 3-dimensional structures. These may be present, for example, in a common interconnect region, such as is described herein. In some cases, for example, the cells may act as an organ, e.g., the cells may be able to emulate one or more functions of a specific organ. In some embodiments, microfluidic devices having such cells or other constructs may be used to study their function, for example, microscopically (e.g., using imaging such as discussed herein), and/or by analyzing media exiting the microfluidic device (e.g., after being exposed to the cells or other constructs), etc. For example, fluid (e.g., cell media) exiting the microfluidic device may be studied to determine proteins, enzymes, nucleic acids, nutrients, waste gases, or the like, e.g., after exposure to the cells or other constructs.

In addition, in some cases, microfluidic devices having such cells or other constructs may be used to determine the effects of agents thereon. For example, cells or other constructs contained within a microfluidic device (e.g., in a common interconnect region) may be exposed to one or more agents that are suspected of being able to interact, and in some cases alter, such cells or other constructs. The agent may be, for example, a pharmaceutical, a drug, a toxin, a biomolecule, or the like. The agent may be supplied to the cells or other constructs, e.g., separately, or along with cell media that is introduced to the microfluidic device. One or more agents may be used.

In addition, in some cases, as discussed, a microfluidic device may contain more than one such system, e.g., as in a plurality of repeat units on a substrate. In some cases, multiple experiments may be performed simultaneously, e.g., exposure to different agents, and/or the same agents at different concentrations, control experiments, etc., may be performed using different repeat units within the microfluidic device. These experiments may be arranged, e.g., systematically or randomly within the microfluidic device.

In addition, certain aspects are generally directed to methods of making microfluidic devices such as those described herein. Additional techniques for making microfluidic devices include those described in U.S. Pat. Apl. Ser. No. 63/412,273, filed on Sep. 30, 2022, entitled “Methods and Systems for Functionalizing Surfaces for Microfluidic Devices or Other Applications,” incorporated herein by reference.

In some embodiments, a fluid may be passed through microfluidic channels within the device. In some cases, such a fluid may contain a precursor of a hydrogel or other scaffold medium, which may be treated (e.g., hardened) to form a hydrogel or other scaffold medium. In some cases, the hydrogel or other scaffold medium may be formed on the polymer or other coating material within a microfluidic channel, which may be more hydrophilic and allow the fluid to contact and readily flow through the microfluidic channel. Thus, certain embodiments such as discussed herein are generally directed to microfluidic channels having a polymer or other coating material, and a hydrogel that is in contact with it, e.g., such that the polymer is positioned between the hydrogel (or other scaffold medium) and one or more walls of the microfluidic channel.

In addition, in certain embodiments, the hydrogel (or other coating material) may be substantively contained within a microfluidic channel, e.g., within a common interconnect region having other microfluidic channels, for example, without the hydrogel being blocked due to pillars, columns, bumps, phaseguides, ridges, or other physical barriers. However, it should be understood that in other embodiments, a hydrogel (or other coating material) may be used in conjunction with pillars, columns, bumps, phaseguides, ridges, or other barriers.

Incorporated herein by reference, each in its entirety, are U.S. Pat. Apl. Ser. No. 63/412,174, filed on Sep. 30, 2022, entitled “Microfluidic Devices Containing Hydrogels, and Techniques for Making and Using”; U.S. Pat. Apl. Ser. No. 63/412,273, filed on Sep. 30, 2022, entitled “Methods and Systems for Functionalizing Surfaces for Microfluidic Devices or Other Applications”; and U.S. Pat. Apl. Ser. No. 63/412,279, filed on Sep. 30, 2022, entitled “Techniques and Systems for Creating Spatially Controlled Fluidic Flows in Surface Functionalized Microfluidic Devices.” Also incorporated herein by reference in its entirety is a provisional patent application filed on Jan. 9, 2023, entitled “Pipette Interface Systems and Methods for Viscous Fluid Injection” U.S. Ser. No. 63/437,955.

The following are each incorporated herein by reference in their entireties: U.S. Provisional Patent Application Ser. No. 63/412,174, filed Sep. 30, 2022, entitled “Microfluidic Devices Containing Hydrogels, and Techniques for Making and Using”; U.S. Provisional Patent Application Ser. No. 63/412,273, filed Sep. 30, 2022, entitled “Methods and Systems for Functionalizing Surfaces for Microfluidic Devices or Other Applications”; U.S. Provisional Patent Application Ser. No. 63/412,279, filed Sep. 30, 2022, entitled “Techniques and Systems for Creating Spatially Controlled Fluidic Flows in Surface Functionalized Microfluidic Devices”; U.S. Provisional Patent Application Ser. No. 63/437,954, filed Jan. 9, 2023, entitled “Edge Effect Systems and Methods for Functionalized Microfluidic Devices”; and U.S. Provisional Patent Application Ser. No. 63/437,955, filed Jan. 9, 2023, entitled “Pipette Interface Systems and Methods for Viscous Fluid Injection.”

In addition, the following patent applications, filed on even date herewith, are incorporated herein by reference in their entireties: a PCT application entitled “Microfluidic Devices Containing Hydrogels, and Techniques for Making and Using”; a PCT application entitled “Methods and Systems for Functionalizing Surfaces for Microfluidic Devices or Other Applications”; a PCT application entitled “Techniques and Systems for Creating Spatially Controlled Fluidic Flows in Surface Functionalized Microfluidic Devices”; and a PCT application entitled “Pipette Interface Systems and Methods for Viscous Fluid Injection.”

Furthermore, the following patent applications, filed on even date herewith, are incorporated herein by reference in their entireties: a US design application entitled “Fluid Channel”; a US design application entitled “Fluid Channel Trench”; a US design application entitled “Well Plate”; a US design application entitled “Fluid Channel”; a US design application entitled “Sample Plate”; and a US design application entitled “Sample Plate Carrier.”

The following examples are intended to illustrate certain embodiments of the present disclosure, but do not exemplify the full scope of the disclosure.

### EXAMPLE 1

This example describes a microfluidic chip having an ECM channel where extracellular matrix (ECM or hydrogel) can be localized and provide a mechanically stable 3D structure for cell cultures. The chip also includes media channels where media can perfuse to sustain cell growth and function.

FIG. 1 shows a top view of a unit cell of the microfluidic chip. In this figure, the sample inlet/outlet ports 1/2 and 3/4 are connected to the ends of the microfluidic channels 11 and 12, respectively, so that hydrogel solution and medium can be introduced directly to the microfluidic channels. This may help to reduce or eliminate the effect of surface tension on liquid flow, e.g., at channel joints or in the microfluidic channels. The ports can be designed so that they can guide the pipet tips towards the microfluidic channels when loading with hydrogel solution. See, e.g., a provisional patent application filed on Jan. 9, 2023, entitled “Pipette Interface Systems and Methods for Viscous Fluid Injection,” U.S. Ser. No. 63/437,955, incorporated herein by reference in its entirety, for additional details on the port.

In addition to the trench, the features at the end of the trench effectively pin the hydrogel solution, preventing it from crossing the trench. The features at the end of the trench may allow it to pin the flow of fluid, e.g., to allow fluid passing through the inlet/outlet ports that allows fluids to directly flow into the channels, e.g., as discussed herein. While in this figure, the features are shown at both ends of the trench, this is by way of example only, and in other embodiments, the features may be present at only one end, and/or different features may be present at different ends of a trench, etc.

FIG. 2 shows a picture of an example microfluidic chip with a solution containing a hydrogel precursor localized in the upper channel after loading. The top piece of the microfluidic chip in this example can be prototyped with medical grade polystyrene (PS), while the bottom piece is an optically transparent thin polystyrene film. In this figure, the hydrogel solution fills top channel 11, but is pinned in place by both the trench 15 and the features at the end of the trench 19, which prevents the solution from entering the bottom channel 12.

In this configuration, it has been shown that when the surface is not functionalized, the hydrogel solution does not flow freely in the microfluidic channel. This behavior is as expected because of the incompatibility of the relatively hydrophilic hydrogel solution with the more hydrophobic polystyrene surface. However, after the microfluidic chip is treated, e.g., with corona plasma treatment, with or without additional polymer coating, the hydrogel solution is able to flow more freely along the ECM channel, while not crossing the trench, as is shown in this figure. The microfluidic chip shown in FIG. 2 was treated with corona plasma for 5.5 minutes with 30 W power and 1.05 mbar chamber pressure. Also, in some embodiments, oxygen plasma may be useful for treatment, e.g., instead of and/or in addition to corona plasma treatment.

The volume and/or flowrate of the solution within the microfluidic channel may be important to facilitate the localization of hydrogel within one channel while preventing the solution from crossing into other channels, e.g., across a trench. Too much fluid will cause the fluid to overflow to the other channel (overfill), while too little fluid may cause incomplete filling of the channel (underfill). Accordingly, a trench may be helpful in certain embodiments to control the proper amount of fluid within a channel without causing it to overflow into other channels.

In this example, the microfluidic chip was designed to be compatible with a 384 well plate. The channel length may be selected to be 9 mm, the channel width can vary from 200 micrometers to 1000 micrometers, and/or the channel height can vary from 120 micrometers to 480 micrometers. The aspect ratio of the channel width versus height may be used to control hydrogel flow and localization within the ECM channel. In some cases, the aspect ratio may be used to control cell conditions in the hydrogel or on the hydrogel/media interface.

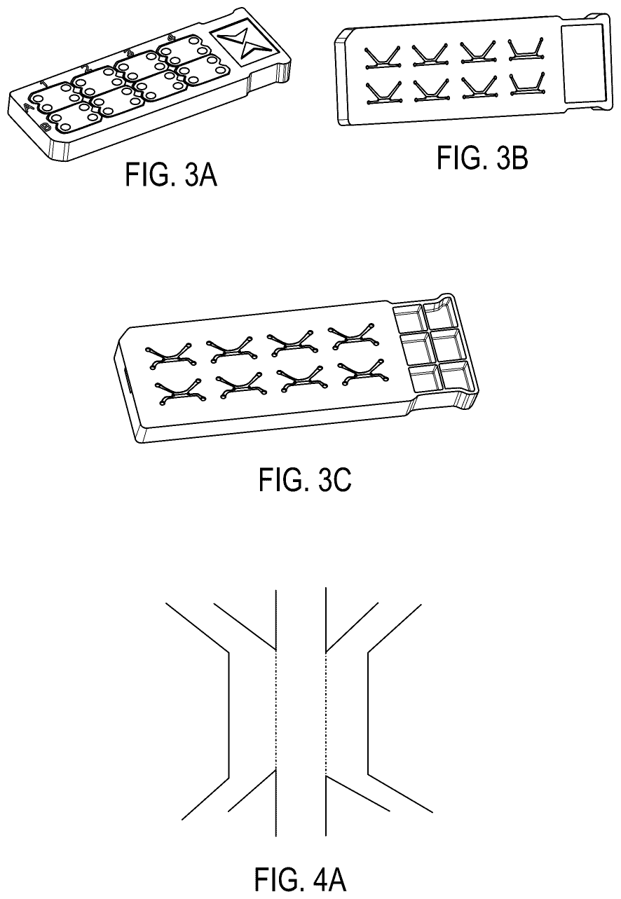

FIG. 3 shows top (FIG. 3A) and bottom (FIG. 3B) views of a microfluidic chip with 8 repeat units, e.g., having a configuration similar to that shown in FIG. 1. Different kinds of hydrogel solutions can be flowed into the microfluidic chips, including collagen I from different sources such bovine and rat, and other hydrogels including any of those described herein, such as other collagens, Matrigel, or the like. Another non-limiting example of such a microfluidic chip is shown in FIG. 3C.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1-53. (canceled)

54. An article, comprising:
a substrate defining a first microfluidic channel having a first inlet and a first outlet, and a second microfluidic channel having a second inlet and a second outlet, the first microfluidic channel and the second microfluidic channel positioned parallel and separated by a trench within a common interconnect region positioned between their respective inlets and outlets, wherein an overhang region is positioned between the trench and the first microfluidic channel.

55. The article of claim 54, wherein the trench has a length longer than a length of an interface between the first microfluidic channel and the second microfluidic channel in the common interconnect region.

56. (canceled)

57. The article of claim 54, wherein the length of the trench is at least 2 mm longer than the length of the interface.

58. (canceled)

59. The article of claim 54, wherein the overhang region defines a distance between an end of the trench and the interface region of at least 2 mm.

60-61. (canceled)

62. The article of claim 54, wherein an end of the trench is in fluid communication with the first microfluidic channel only via the trench.

63. (canceled)

64. The article of claim 54, wherein the trench is substantially straight.

65. The article of claim 54, wherein the trench has a cross-sectional dimension of at least 100 micrometers.

66-68. (canceled)

69. The article of claim 54, wherein the trench has a depth of at least 100 micrometers.

70. (canceled)

71. The article of claim 54, wherein the common interconnect region has a length defined where the first microfluidic channel and the second microfluidic channel are positioned parallel of at least 3 mm.

72-75. (canceled)

76. The article of claim 54, wherein the first microfluidic channel contains a hydrogel.

77-105. (canceled)

106. A method, comprising:
providing a substrate defining a first microfluidic channel having a first inlet and a first outlet, and a second microfluidic channel having a second inlet and a second outlet, the first microfluidic channel and the second microfluidic channel positioned parallel within a common interconnect region positioned between their respective inlets and outlets; and
passing a fluid through the first microfluidic channel from the inlet towards the outlet, through the common interconnect region, wherein the fluid is prevented from entering the second microfluidic channel via a trench in a wall of the common interconnect region, wherein the trench has a length longer than a length of an interface between the first microfluidic channel and the second microfluidic channel in the common interconnect region.

107-132. (canceled)

133. An article, comprising:
a substrate defining a microfluidic interconnect region defined by a first microfluidic channel, a second microfluidic channel parallel to the first microfluidic channel, and one or more overhang regions positioned between the first microfluidic channel and the second microfluidic channel.

134. The article of claim 133, wherein the first microfluidic channel contains a hydrogel.

135. The article of claim 134, wherein the hydrogel fills the first microfluidic channel but not the second microfluidic channel.

136. The article of claim 134, wherein the hydrogel partially fills the common interconnect region.

137. The article of claim 134, wherein at least 20% of any cross-section of the common interconnect region is not filled with the hydrogel.

138. The article of claim 134, wherein at least a portion of the hydrogel in the first microfluidic channel is exposed to the second microfluidic channel.

139. The article of claim 134, wherein the portion of the hydrogel exposed to the second microfluidic channel does not contain an interface material separating the hydrogel from the second microfluidic channel.

140. The article of claim 133, wherein the substrate has dimensions of (75 mm+/−2 mm)×(26 mm+/−2 mm).

141. The article of claim 133, wherein the substrate has dimensions of (128 mm+/−5 mm)×(85 mm+/−5 mm).
